(19) United States
(12) Patent Application Publication
(10) Pub. No.: US 2009/0254033 A1
(43) Pub. Date: Oct. 8, 2009

(54) Catheter and catheter kit

(76) Inventors: Masaru Suzuki, Kanagawa (JP); Kazuhito Yabuki, Saitama (JP)

Correspondence Address: Kubotera & Associates, LLC, 200 Daingerfield Rd, Suite 202, Alexandria, VA, 22314, US

(21) Appl. No.: 11/991,138

(22) PCT Filed: May 31, 2006
(86) PCT No.: PCT/JP2006/310857

§ 371 Date: Feb. 28, 2008

(30) Foreign Application Priority Data
Sep. 7, 2005 (JP) 2005-259272

(51) Int. Cl. A61M 25/10

(52) U.S. Cl. 604/97.02

(57) ABSTRACT

A catheter includes an insertion portion at a distal end of an introduction tube that is thinned to an utmost limit, so that the catheter can be easily inserted and operated with patient's pain alleviated, and a catheter kit includes the catheter. An inflatable and deflatable balloon (50) for prevention of undesired come-out is installed at the distal end of the introduction tube (20). The balloon (50) has a rubber balloon-like shape, and is attached to a distal end part (27) of the introduction tube (20) so as to project from the distal end part (27).

### TECHNICAL FIELD

The present invention relates to a catheter for introducing nutrition, or the like, into an inside of an internal organ. The catheter includes an inflatable and deflatable balloon installed at a distal end of an introduction tube thereof for preventing the catheter from coming off. The present invention also relates to a catheter kit.

### BACKGROUND ART

As an example of the prior art, an invention disclosed in Published Japanese Patent Application No. 2004-41349 (JP 2004-41349 A) is available.

Specifically, the technology disclosed in the document involves a balloon which is attached to a distal end part of an introduction tube, i.e., a main body of a catheter. In the catheter, when the balloon is inflated, the balloon juts out ahead of a distal end of the main body of the catheter, and is adhered to the main body of the catheter so as to be overlapped with the main body of the catheter without being turned up.

### DISCLOSURE OF THE INVENTION

However, in the prior art, although the balloon is overlapped, as a whole, with the distal end part of the introduction tube, i.e., the main body of the catheter, without being turned up, a wall thickness of a portion of the catheter to be inserted increases as a whole. Accordingly, at the time of insertion of the catheter, the distal end part becomes larger than the introduction tube, thereby making the insertion difficult. Further, handling of the catheter becomes cumbersome, and a patient may be subject to pain.

The present invention has been made in view of such problems of the prior art, and an object of the present invention is to provide a catheter having a small insertion portion at a distal end of an introduction tube thereof, so that the catheter can be easily inserted and operated while alleviating patient's pain. Another object of the present invention is to provide a catheter kit.

The subject matters of the present invention to achieve the above purpose are disclosed in the following respective aspects of the present invention:

According to a first aspect of the present invention, a catheter for introducing nutrition, or the like, into an inside of an internal organ, includes an inflatable and deflatable balloon installed at a distal end of an introduction tube thereof for preventing the catheter from coming off, wherein

said introduction tube includes an injection path, and an air passage which guides air to the balloon; and

said balloon has a rubber balloon-like shape; is attached to a distal end part of the introduction tube such that the balloon is projected from the distal end part; when the balloon is inflated, is located ahead of an outlet port at the distal end part of the introduction tube to form an outlet path with which the outlet port communicates; and has a peripheral edge part including a catch part which, when the balloon is deflated, can catch an insertion auxiliary rod inserted through the injection path of the introduction tube.

According to a second aspect of the present invention, a catheter kit includes the catheter according to the first aspect, wherein the catheter is combined with the insertion auxiliary rod.

The present invention functions as follows.

The catheter serves to introduce nutrition, or the like, into the inside of an internal organ, and therewith, nutrition, or the like, is injected into the inside of the internal organ from a fistula, such as a gastric fistula, an intestinal fistula, or the like, through the introduction tube. The balloon having a rubber balloon-like shape that is installed at the distal end part of the introduction tube is deflated at the time of loading the catheter, and is forced to be projected from the distal end part of the introduction tube such that the balloon is deformed into an awl-like shape, having a thinned tip part. Accordingly, it is possible to easily insert the distal end part of the catheter into the inside of the internal organ from the gastric fistula or the intestinal fistula. After the insertion, the balloon is inflated to a size larger than the gastric fistula or the intestinal fistula for serving as a measure against undesired coming-out of the introduction tube.

Specifically, when the catheter is to be loaded in the patient, the air in the balloon is drained through the air passage provided in parallel with the injection path in the introduction tube. As a result, the balloon is deflated as if the balloon is a rubber balloon from which the air is drained.

In this state, the insertion auxiliary rod is inserted into the injection path in the introduction tube until the insertion auxiliary rod is caught by the catch part in the peripheral edge part of the balloon. Then, the insertion auxiliary rod further advances, so that the balloon is projected from the distal end part of the introduction tube, being deformed into an awl-like shape, having a thinned tip part. In this state, the distal end part of the introduction tube can be inserted into the inside of the internal organ from the gastric fistula or the intestinal fistula with smoothness and gentleness. Accordingly, it is possible to make the loading easy, greatly alleviate the burden on the patient, and eliminate the possibility of occurrence of a medical accident.

Forcing in/out the air into/from the balloon is performed through the air conduction plug part on an injection end side of the introduction tube. The air conduction plug part connected to the end part of the air passage for guiding the air into the balloon causes the air passage to be in communication with the outside only when a syringe is loaded thereonto.

When the air in the balloon is to be drained, the syringe is used to suck up the air until the balloon is completely collapsed. In this state, the distal end part of the introduction tube is inserted into the inside of the internal organ in the manner described above.

In the state in which the catheter is loaded, the balloon having a rubber balloon-like shape is located ahead of the outlet port of the introduction tube at the distal end thereof, and from the outlet path which communicates with this outlet port, nutrition, or the like, is introduced into the inside of the internal organ. In addition, even if the balloon contacts with the inside wall of the internal organ, the introduction tube does not be directly contact therewith, and because the balloon is soft, having a rubber balloon-like shape, there is little possibility that the balloon may have a detrimental effect on the internal organ.

The catheter kit which combines the catheter with the insertion auxiliary rod can be conveniently utilized for medical devices as it is. Further, the catheter kit is a carefully and deliberately designed instrument, thereby being free from operational error, and assuring safety.

### BRIEF DESCRIPTION OF THE DRAWINGS

FIG. 1 is an explanatory drawing illustrating a catheter pertaining to an embodiment of the present invention in cross section;

FIG. 2 is a front view illustrating the catheter pertaining to the embodiment of the present invention with a balloon being inflated;

FIG. 3 is an explanatory drawing illustrating the catheter pertaining to the embodiment of the present invention in a state that the catheter is inserted from a fistula and the balloon is inflated; and

FIG. 4 is an explanatory drawing illustrating the catheter pertaining to the embodiment of the present invention in a state that the catheter is inserted from a fistula and the insertion auxiliary rod is withdrawn.

### BEST MODE FOR CARRYING OUT THE INVENTION

Hereinbelow, with reference to the drawings, one exemplary embodiment of the present invention will be described.

The figures show one embodiment of the present invention.

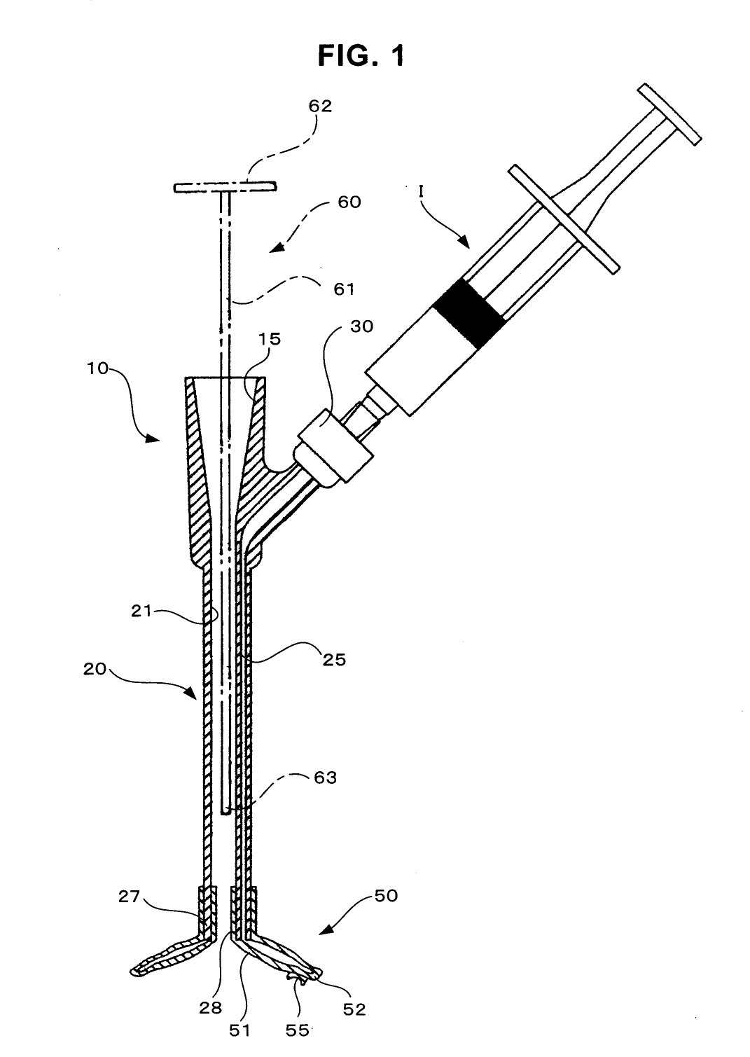

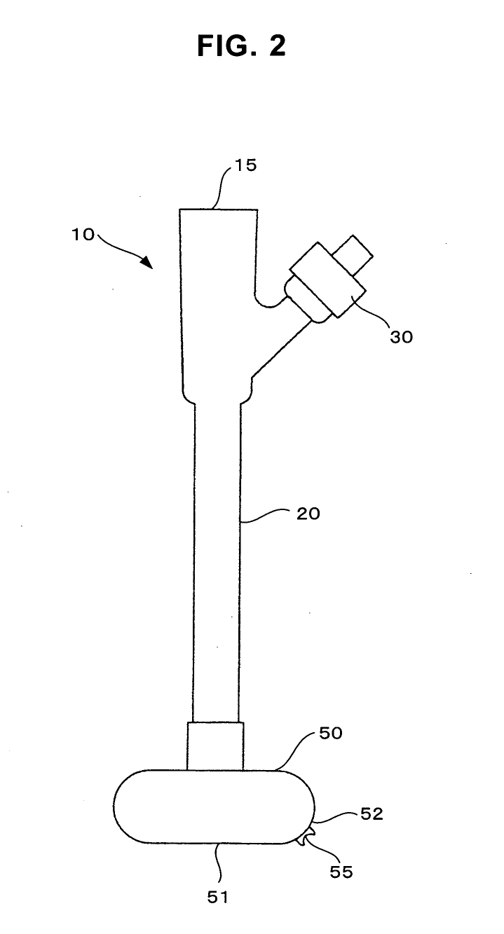

FIG. 1 shows a cross section of a catheter 10 with a syringe I being loaded thereonto, and FIG. 2 is an outline view of the catheter 10. As shown in the figures, the catheter 10 includes an inflatable and deflatable balloon 50 for come-out prevention that is installed at the distal end of an introduction tube 20, and the catheter 10 is used for introduction of nutrition, or the like, into the inside of an internal organ.

On the inlet side of the introduction tube 20 of the catheter 10, an injection port 15 with a flared opening is formed so that nutrition, or the like, can be injected through the injection port 15.

The introduction tube 20 includes an injection path 21, and an air passage 25 which guides air to the balloon 50. The air passage 25 is extremely thin, but thick enough to allow air to be passed therethrough.

Adjacently to the injection port 15 on the injection end side of the introduction tube 20, an air conduction plug part 30 is provided as if it were branched from the introduction tube 20 in the shape of a letter Y. The air conduction plug part 30 is connected to the end part of the air passage 25 which guides air to the balloon 50, and is configured such that it allows the air passage 25 to communicate with the outside only when the syringe I is loaded. For example, the air conduction plug part 30 has a check valve which, when the syringe I is inserted, provides a communicated state to allow air to be forced in/out.

The balloon 50 has a rubber balloon-like shape, being attached to a distal end part 27 of the introduction tube 20 such that it is projected from the distal end part 27. In other words, an elastic thin film of the balloon 50 is jointed to the distal end part 27 of the injection path 21 such that both edge parts thereof sandwich the distal end part 27, and when the air is drained, the balloon 50 takes such a configuration as that which would be taken by a doughnut formed around the distal end part 27 and collapsed. The balloon 50 can be deformed such that it is projected from the distal end of the introduction tube 20 like an awl as a whole.

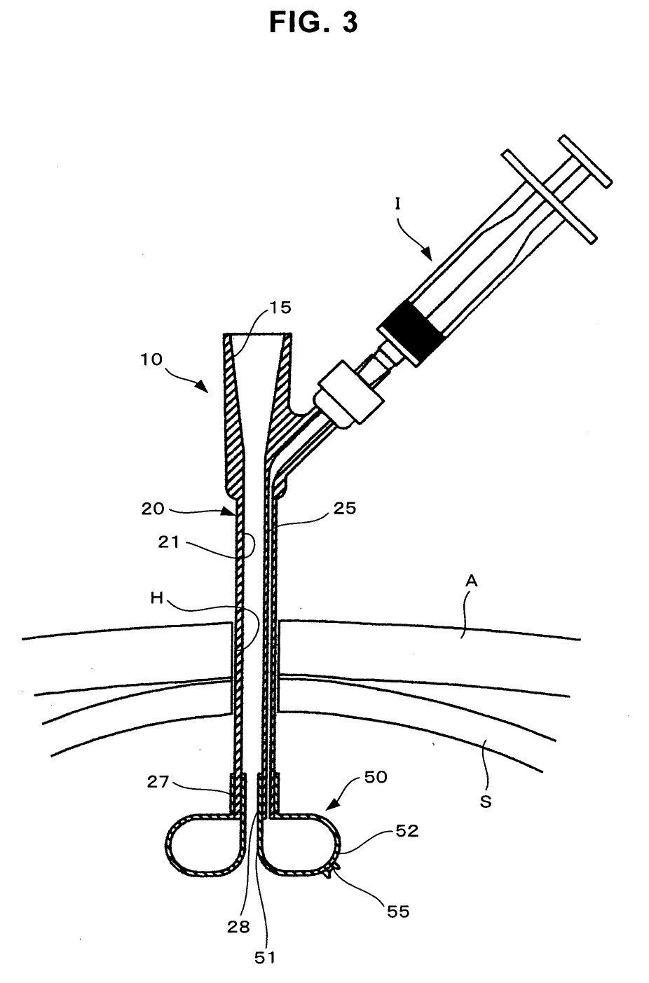

As can be seen from FIG. 2 and FIG. 3, the balloon 50 is adapted such that, when inflated, it has a doughnut-like configuration located ahead of the outlet port 28 at the distal end of the introduction tube 20, an outlet path 51 with which the outlet port 28 communicates being formed in the central portion of the balloon 50.

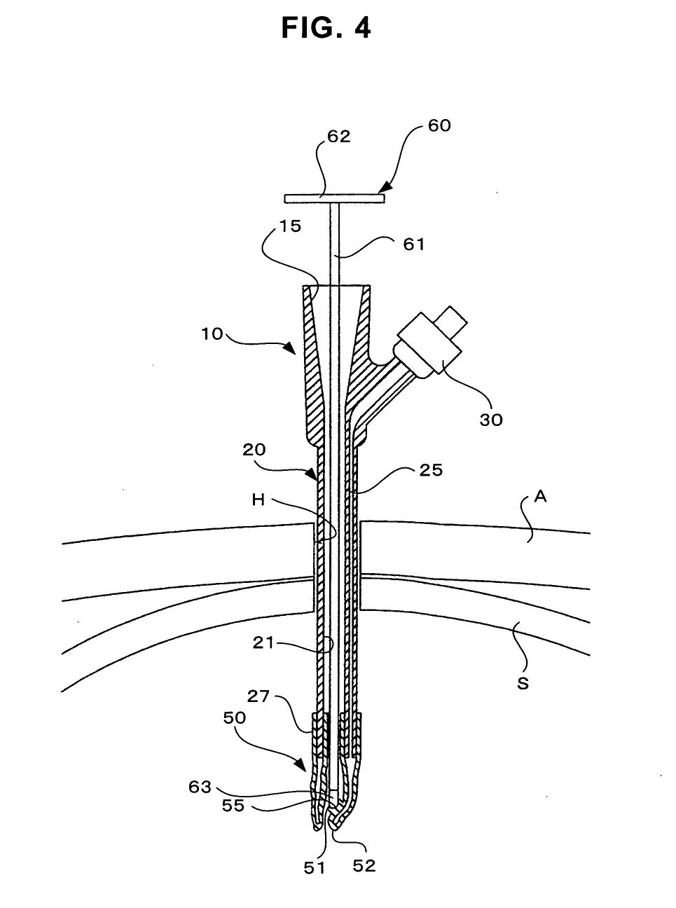

As shown in FIG. 4, the peripheral edge part 52 of the balloon 50 includes a catch part 55 which, when the balloon 50 is deflated, can catch an insertion auxiliary rod 60 inserted through the injection path 21 of the introduction tube 20.

The insertion auxiliary rod 60 is made up of a rod part 61 provided with a holding head part 62 and a distal end 63 which can be fitted into the catch part 55. On the other hand, the catch part 55 is formed such that, as can be seen from FIG. 4, it can catch the distal end part 63 of the insertion auxiliary rod 60, and then will not be out of place even if angularly displaced.

FIG. 4 illustrates the state just prior to the balloon 50 being deformed into an awl-like shape, and from such state, pressing the holding head part 62 to stick out the distal end part 63 will also pull down the portion on the opposite side that is turned up in FIG. 4, resulting in the balloon 50 being deformed into an awl-like shape, having a thinned tip part, as a whole.

Next, the function of the balloon 50 will be described.

The catheter 10 serves to introduce nutrition, or the like, into the inside of an internal organ, and therewith, nutrition, or the like, is injected into the inside of an internal organ from a fistula H, such as a gastric fistula, an intestinal fistula, or the like, through the introduction tube 20.

Specifically, as shown in FIG. 4, a fistula H is opened in the abdominal wall A and the gastric wall S, and the catheter 10 is inserted into the fistula H from the distal end part.

The balloon 50 having a rubber balloon-like shape that is installed at the distal end of the introduction tube 20 is deflated at the time of loading the catheter 10, and is forced to be projected from the distal end part 27 of the introduction tube 20 such that the distal end part 27 of the catheter 10 can be inserted into the inside of the internal organ from the fistula H, being deformed into an awl-like shape, having a thinned tip part. After the insertion, as shown in FIG. 3, the balloon 50 is inflated to a size larger than the gastric fistula or the intestinal fistula for serving as a measure against undesired coming-out of the introduction tube 20.

As shown in FIG. 1, before the catheter 10 is loaded in the patient, the syringe I is inserted into the air conduction plug part 30 to fully drain the air in the balloon 50 through the air passage 25 which is provided in parallel with the injection path 21 in the introduction tube 20, whereby the balloon 50 is deflated as if it were a rubber balloon from which the air has been drained. Because the balloon 50 inflated forms an outlet path 51 in the central portion thereof, it, when deflated, takes such a configuration as that which would be taken by a doughnut collapsed.

When the air in the balloon 50 is to be drained, the syringe I is used to suck up the air until the balloon 50 is completely collapsed. In this state, the distal end of the introduction tube 20 is inserted into the inside of the internal organ in the manner as described above.

Forcing in/out the air into/from the balloon 50 is performed through the air conduction plug part 30 on the injection end side of the introduction tube 20. The air conduction plug part 30 connected to the end part of the air passage 25 for guiding the air into the balloon 50 causes the air passage 25 to be in communication with the outside only when the syringe I is loaded to be inserted thereinto.

With the air having been drained from the balloon 50, inserting the insertion auxiliary rod 60 into the injection path 21 in the introduction tube 20 will cause the distal end part 63 of the insertion auxiliary rod 60 to be fitted into the catch part 55 in the peripheral edge part 52 of the balloon 50 to be caught thereby. Further advancing the insertion auxiliary rod 60 by pressing it will cause the balloon 50 to be projected from the distal end part 27 of the introduction tube 20, being deformed into an awl-like shape, having a thinned tip part.

In this state, the distal end part 27 of the introduction tube 20 can be inserted into the inside of the internal organ from the gastric fistula or the intestinal fistula with smoothness and gentleness, which makes the loading easy, greatly alleviates the burden on the patient, and eliminates the possibility of occurrence of a medical accident.

FIG. 4 illustrates the state in which the insertion auxiliary rod 60 has started to be slightly withdrawn, and if the insertion auxiliary rod 60 were stuck out, a part of the balloon 50 would be projected along the rod part 61 of the insertion auxiliary rod 60, resulting in the balloon 50 being deformed into an awl-like shape, having a thinned tip part, as a whole.

From the state in FIG. 4, the insertion auxiliary rod 60 is withdrawn, and as shown in FIG. 3, the syringe I is loaded onto the air conduction plug part 30 to force in the air into the balloon 50 through the air passage 25 for inflating the balloon 50. The balloon 50 inflated has a doughnut-like configuration in which an outlet path 51 is formed in the central portion. To the injection port 15 of the catheter 10, a nutrition tube, or the like, is connected.

In the state in which the catheter 10 is loaded, the balloon 50 having a rubber balloon-like shape is located ahead of the outlet port 28 at the distal end of the introduction tube 20, and from the outlet path 51 which communicates with this outlet port 28, nutrition, or the like, is introduced into the inside of the internal organ. In addition, even if the balloon 50 should be contacted with the inside wall of the internal organ, the introduction tube 20 will not be directly contacted therewith, and because the balloon 50 is soft, having a rubber balloon-like shape, there is no possibility that the balloon 50 may have a detrimental effect on the internal organ.

The catheter kit which combines the catheter 10 with the insertion auxiliary rod 60 can be conveniently utilized for medical devices as it is, and it is a carefully and deliberately designed instrument, thus it is free from operational error, assuring safety.

### INDUSTRIAL APPLICABILITY

According to the catheter and catheter kit pertaining to the present invention, at the time of loading the catheter, the balloon is forced to be projected from the distal end of the introduction tube, being deformed into an awl-like shape, having a thinned tip part, and then the distal end part of the introduction tube is inserted into the inside of an internal organ from the gastric fistula or the intestinal fistula, which allows insertion to be made smooth and gentle, makes the loading easy, greatly alleviates the burden on the patient, and eliminates the possibility of occurrence of a medical accident.

In the state in which the catheter is loaded, the balloon having a rubber balloon-like shape is located ahead of the outlet port at the distal end of the introduction tube, thus, even if the balloon should be contacted with the inside wall of the internal organ, the introduction tube will not be directly contacted therewith, and because the balloon is soft, having a rubber balloon-like shape, there is no possibility that the balloon may have a detrimental effect on the internal organ.

If the catheter is combined with the insertion auxiliary rod to provide a catheter kit, the catheter kit can be conveniently utilized for medical devices as it is, and it is a carefully and deliberately designed instrument, thus it is free from operational error, assuring safety.

What is claimed is:

1. A catheter (10) for introducing nutrition, or the like, into an inside of an internal organ, comprising:
an inflatable and deflatable balloon (50) installed at a distal end of an introduction tube (20) for preventing the catheter from coming off, wherein
said introduction tube (20) includes an injection path (21), and an air passage (25) which guides air to the balloon (50); and
said balloon (50) has a rubber balloon-like shape; is attached to a distal end part (27) of the introduction tube (20) so that the balloon is projected from the distal end part (27); when the balloon (50) is inflated, the balloon is located ahead of an outlet port (28) at the distal end part of the introduction tube (20) to form an outlet path (51) with which the outlet port (28) communicates; and the balloon has a peripheral edge part (52) including a catch part (55) which, when the balloon (50) is deflated, can catch an insertion auxiliary rod (60) inserted through the injection path (21) of the introduction tube (20).

2. A catheter kit, wherein the catheter (10) according to claim 1 is combined with the insertion auxiliary rod (60).
